(19) United States
(12) Patent Application Publication
(10) Pub. No.: US 2013/0046086 A1
(43) Pub. Date: Feb. 21, 2013

(54) METHOD FOR SEPARATING CARBON NANOTUBES

(71) Applicant: Steven Raymond Lustig, Landenberg, PA (US)

(72) Inventor: Steven Raymond Lustig, Landenberg, PA (US)

(73) Assignee: E. I. DU PONT DE NEMOURS AND COMPANY, Wilmington, DE (US)

(21) Appl. No.: 13/510,422

(22) PCT Filed: Nov. 19, 2010
(86) PCT No.: PCT/US10/57360

§ 371 Date: May 17, 2012

Related U.S. Application Data
(60) Provisional application No. 61/262,778, filed on Nov. 19, 2009.

(51) Int. Cl. C07H 21/00

(52) U.S. Cl. 536/25.41

(57) ABSTRACT

This invention relates methods and apparatus to separate charged species such as carbon nanotubes by their electrical conductivity. Carbon nanotubes of varying electrical conductivity may be separated by hybridizing the nanotubes with nucleic acids such as DNA and then passing a dispersion of the hybrids through an electrochemical cell with an electrical potential on the working electrode which absorbs or desorbs nanotubes of a desired electrical conductivity.

This application claims priority under 35 U.S.C. §119(e) from, and claims the benefit of, U.S. Provisional Application No. 61/262,778, filed Nov. 19, 2009, which is by this reference incorporated in its entirety as a part hereof for all purposes.

### TECHNICAL FIELD

This invention relates to methods and apparatus for separating a mixture of charged species, such as a mixture of carbon nanotubes (“CNTs”) dispersed in a mobile phase such as a liquid.

### BACKGROUND

CNTs have been the subject of intense research since their discovery in 1991. CNTs possess unique properties such as small size and electrical conductivity, which make them suitable in a wide range of applications, including use as structural materials in molecular electronics, nanoelectronic components and field emission devices. CNTs may be either single-walled carbon nanotubes (SWNTs) or multi-walled carbon nanotubes (MWNTs), and have diameters in the nanometer range.

Depending on their atomic structure, CNTs may have either metallic or semiconductor properties, and these properties, in combination with their small dimensions make them particularly attractive for use in fabrication of nano-scale devices. A major obstacle to accomplishment of such objective has been the diversity of tube diameters, chiral angles, and aggregation states in nanotube samples obtained from the various preparation methods. Aggregation is particularly problematic because CNTs are essentially highly polarizable, smooth-sided fullerene tubes that readily form parallel bundles or ropes with a large van der Waals binding energy. This bundling perturbs the electronic structure of the tubes, and it confounds various attempts to separate the tubes by size or type or to use them as individual macromolecular species.

Although there have been many reports on producing suspensions enriched in individual fullerene tubes, including those referenced as follows:
Liu et al, Science 280, 1253 (1998);
O'Connell et al, Chem. Phys. Lett. 342, 265 (2001);
Bandow et al, J. Phys. Chem. B 101, 8839 (1997);
Chen et al, Science 282, 95 (1998);
Duesberg et al, Appl. Phys. A 67, 117 (1998);
Dalton et al, J. Phys. Chem. B 104, 10012 (2000);
Dalton et al, Synth. Metals 121, 1217 (2001);
Bandyopadhyaya et al, Nano Lett. 2, 25 (2002);
   available samples are typically still dominated by small nanotube bundles. O'Connell et al [Science 297, 593 (2002)] have described a method of tube separation based on vigorous treatment with a sonicator followed by centrifugation, primarily yielding individual fullerene nanotubes in aqueous micellar suspensions. Also described are processes (O'Connell et al, Chem. Phys. Lett., 342, 265, 2001; and WO 02/076888) for the solubilization of carbon nanotubes in water by association with selected polymers, although different polymers produced differing degrees of success.

Once CNTs are in a dispersed form suitable for further manipulation, a desirable next step is self-assembly of the nanotubes on a solid substrate. Associating oligonucleotides to CNTs would allow one to use biomolecular techniques for the positioning of the nanotubes on a substrate. Williams et al (AIP Conf. Proc. 663, 444, 2002) have covalently coupled peptide nucleic acid oligomers to CNTs and then hybridized this construct to DNA. However, DNA was not directly attached to the nanotubes, nor was dispersion of nanotube bundles observed.

Zheng et al (US 2004/0132072) disclose a method separating CNTs of varying electrical conductivity by hybridizing the CNTs with DNA and passing the hybrids through a liquid chromatography column which is exposed to a solution with a varying concentration of a salt. It was observed in Zheng that DNA forms stable hybrids with SWNTs, and that ion-exchange chromatography could be used to separate the hybrids based on the chirality of the SWNTs within the hybrids. This provided a way to separate SWNTs according to their chirality. This ion-exchange chromatography method requires the use of an ion-exchange chromatography column and a concentrated salt solution to make the separation. However, the yield of this method, particularly for long hybrids (which may be on the order of near microns in length), may be adversely affected because long hybrids tend to get trapped within the ion exchange chromatography column. Since DNA is very expensive, loss of SWNTs and the surrounding DNA is not desirable. The separated elutions of hybrids are also contaminated with a concentrated salt solution, and further purification steps are thus required.

Known production processes for single-walled carbon nanotubes produce a mixture of nanotube chiralities having different metallic and semiconductive electrical bandgaps, conductivities and diameters. Some potential applications require one conductivity vs. another, alternatively one bandgap vs. another, and/or alternatively one diameter vs. another. A mixture of nanotubes may thus be undesirable.

A need thus remains for methods and apparatus for the facile, high-yield and inexpensive separation of bundled carbon nanotubes, as well as other charged species, which will enhance their ready and economical use in the fabrication of nano-scale devices, sensors and other applications.

### SUMMARY

This invention relates to methods and apparatus for separating a mixture of charged species, such as a mixture of carbon nanotubes dispersed in a mobile phase such as a liquid.

In one embodiment, this invention provides a method of partitioning a population of charged species by (a) providing a dispersion of the charged species in a liquid, (b) contacting the liquid dispersion with a first electrode to which is applied a voltage selected to attract and adsorb to the electrode a portion of the species contained in the liquid dispersion, (c) desorbing from the electrode some or all of the adsorbed species to provide a first fraction of species, and (d) extracting the first fraction of species from the population.

In alternative embodiments, one or more charged species may be a hybrid formed from a carbon nanotube and a nucleic acid molecule, and bundled carbon nanotubes may be contacted with nucleic acid molecules to form the dispersion of charged species.

In further embodiments, the voltage of the first electrode may be adjusted to desorb adsorbed species from the first electrode; the voltage of the first electrode may be adjusted in a time-varying pattern; or the voltage of the first electrode may be adjusted in discrete steps or in a continuous ramp. Other alternatives include embodiments wherein the composition of the liquid dispersion is adjusted to desorb adsorbed species from the first electrode; and/or wherein a stream of the liquid dispersion is flowed past the first electrode to contact the liquid dispersion with the first electrode, and the velocity of the stream is adjusted to desorb adsorbed species from the first electrode.

Yet other alternatives include embodiments wherein the species are negatively charged, the species are positively charged, the first electrode is negatively charged, and/or the first electrode is positively charged. Other alternative embodiments include determining the weight of the portion of species that are adsorbed on the first electrode.

Other alternative embodiments include contacting the liquid dispersion with a second electrode to which is applied a voltage selected to attract and adsorb to the second electrode the same or a different portion of the species contained in the liquid dispersion than is attracted to the first electrode.

Other alternatives include embodiments wherein the voltage applied to the second electrode attracts and adsorbs on the electrode a different portion of the species contained in the liquid dispersion than is attracted to the first electrode.

Other alternatives include embodiments wherein the voltage applied to the second electrode attracts and adsorbs on the second electrode the same portion of the species contained in the liquid dispersion that is attracted to the first electrode; wherein the voltage of the first electrode is adjusted by a first amount to provide the first fraction of species; and wherein the voltage of the second electrode is adjusted by a second amount to provide a second fraction of species.

Other alternative embodiments include analyzing the first fraction of species to determine a property thereof.

Other alternatives include embodiments wherein the property determined is an optical, chemical or electrical property of the species.

Other alternatives include embodiments wherein the desorbed species are passed through a diode array detector to determine a property thereof.

Other alternative embodiments include directing the first fraction of species to a first preselected container in a fractionator to extract the first fraction of species from the population.

Other alternative embodiments include evaporating or filtering the liquid dispersion containing the first fraction of species to recover the species from the liquid.

Other alternative embodiments include immobilizing the first fraction of species on a solid support.

Other alternative embodiments include collecting the first fraction of species; extracting from the population and collecting some or all of the species that are not attracted to the first electrode to provide a third fraction of species; analyzing the first fraction of species to determine a property thereof that identifies them as enriched in Type I species; analyzing the third fraction of species to determine a property thereof that identifies them as enriched in Type III species; contacting the first fraction again with the first electrode to provide an adsorbed/desorbed fraction that is further enriched in Type I species; and repeatedly contacting the desorbed fraction with the first electrode to provide fractions that are increasingly enriched in Type I species.

Other alternative embodiments include collecting the first fraction of species; extracting from the population and collecting some or all of the species that are not attracted to the first electrode to provide a third fraction of species; analyzing the first fraction of species to determine a property thereof that identifies them as enriched in Type I species; analyzing the third fraction of species to determine a property thereof that identifies them as enriched in Type III species; contacting the third fraction again with the first electrode to provide an non-attracted fraction that is further enriched in Type III species; and repeatedly contacting the non-attracted fraction with the first electrode to provide fractions that are increasingly enriched in Type III species.

This invention utilizes a method for dispersing a population of CNTs, such as bundled carbon nanotubes, that involves contacting the bundled nanotubes with a stabilized solution of nucleic acid molecules. It has been found that nucleic acids are very effective in dispersing the nanotubes, and forming nanotube-nucleic acid complexes based on non-covalent interactions between the nanotube and the nucleic acid molecule.

This invention, in both its method and apparatus aspects, also relates to the separation of individual species from a mixture of species suspended in a mobile phase. The method and apparatus are particularly useful since a major obstacle to the manipulation and use of carbon nanotubes as structural materials has been their poor solubility and their tendency to aggregate in bundles or clusters.

In particular, carbon nanotubes of varying electrical conductivity or bandgap or diameter may be separated by hybridizing the nanotubes with DNA and then passing a dispersion of the hybrids through an electrochemical cell with an electrical potential on a working electrode that adsorbs or desorbs or allows by-pass of nanotubes of a selected electrical conductivity, band gap or diameter.

The method and apparatus of the present invention not only separates metallic from semiconducting CNTs, but also separates semiconducting CNTs according to the varying chiralities thereof. This is important because various chiralities of semiconducting CNTs differ significantly by their electronic band-gap. Other processes that separate metallic from semiconducting types of CNTs are not able to separate the semiconducting CNTs by bandgap, which further differentiates their electronic properties as that relates to their desired use. The method and apparatus of the present invention also separates metallic CNTs of differing diameter.

### BRIEF DESCRIPTION OF THE DRAWINGS AND FIGURES

FIG. 1 shows a schematic diagram of one version of an electrochemical cell suitable for use in this invention.

### DETAILED DESCRIPTION

Various embodiments of this invention provide methods and apparatus for separating a mixture individual charged species from a mixture of charged species carried in a mobile phase. In various embodiments, the charged species that are separated (such as CNTs) can be separated according to differences among them of conductivity and/or chirality. The methods of this invention may be performed by use of the apparatus of this invention, and the apparatus of this invention may perform methods of separation including those of this invention.

In the various embodiments hereof in which the charged speices that are separated are CNTs, there is provided for use herein a hybrid that is formed from CNTs and nucleic acid molecules. A CNT suitable for use herein to form a hybrid is generally a hollow article composed primarily of carbon atoms that has a narrow dimension (essentially its diameter) of about 0.5 to about 10 nm, or about 1 to about 2 nm, and a longer dimension (its length) such that the ratio of the longer dimension to the narrow dimension, i.e. the aspect ratio, is at least about 5. In general, the aspect ratio is between about 10 and about 2000. The CNTs are comprised primarily of carbon atoms but may be doped with other elements such as various metals.

As noted above, CNTs as used herein can be either SWNTs or MWNTs. A MWNT includes, for example, several concentric tubular layers each having a different diameter. The smallest diameter tube is thus encapsulated by a larger diameter tube, which in turn is encapsulated by another larger diameter tube. A SWNT, on the other hand, includes only one tube. A CNT may be classified according to its chirality, which is defined by a vector (m,n) wherein the components m,n are integers and refer to the atomic pattern of carbons in the nanotube. CNTs as used herein may include a mixture of conducting nanotubes and semiconducting nanotubes, or a mixture of conducting nanotubes, or a mixture of semiconducting nanotubes. These mixture may exhibit a range of chiralities and conductivities.

CNTs suitable for use herein may be produced by a variety of methods, and are additionally commercially available. Methods of CNT synthesis include laser vaporization of graphite [Thess et al, Science 273, 483 (1996)]; arc discharge [Journet et al, Nature 388, 756 (1997)]; and the HiPCo (high pressure carbon monoxide) process [Nikolaev et al, Chem. Phys. Lett. 313, 91-97 (1999)]. Chemical vapor deposition (CVD) can also be used in producing carbon nanotubes [Kong et al, Chem. Phys. Lett. 292, 567-574 (1998); Kong et al, Nature 395, 878-879 (1998); Cassell et al, J. Phys. Chem. 103, 6484-6492 (1999); and Dai et al, J. Phys. Chem. 103, 11246-11255 (1999)].

CNTs may additionally be grown via catalytic processes both in solution and on solid substrates [Yan Li et al, Chem. Mater., 2001; 13(3) 1008-1014; Franklin et al, Adv. Mater. 12, 890 (2000); and Cassell et al, J. Am. Chem. Soc. 121, 7975-7976 (1999)]. CNTs made by the HiPCo process, either purified or unpurified, may be purchased from CNI (Houston, Tex.).

When separating CNTs, as the charged species herein, CNTs can be dispersed in a mobile phase by forming hybrids of CNTs and nucleic acid molecules, and the dispersion of bundled CNTs can thus obtained by contacting them with a stabilized solution of nucleic acid molecules to form such hybrids. A hybrid as formed herein is a CNT-nucleic acid complex, which is a composition that includes a CNT loosely associated with at least one nucleic acid molecule. Typically the association between the CNT and the nucleic acid molecule is by van der Waals bonds or other non-covalent means.

A nucleic acid molecule is a polymer of RNA, DNA or peptide nucleic acid (PNA) that is single- or double-stranded, optionally containing synthetic, non-natural or altered nucleotide bases. A nucleic acid molecule in the form of a polymer of DNA may be composed, for example, of one or more segments of cDNA, genomic DNA or synthetic DNA. A peptide nucleic acid is a material having stretches of nucleic acid polymers linked together by peptide linkers. Peptide nucleic acids (PNA) possess the double functionality of both nucleic acids and peptides. When desired, the denaturation of DNA or other nucleic acid molecules by use of a denaturant.

A stabilized solution of nucleic acid molecules as used to form hybrids is a solution of nucleic acid molecules that are solubilized and in a relaxed secondary conformation. A mobile phase in which hybrids reside is a fluid that carries dispersed carbon nanotubes, nucleic acid molecules and hybrids formed therefrom, and is flowable through an apparatus of this invention and is flowable for the purpose of performing the methods of this invention. A mobile phase may be an aqueous fluid or may be pure water. The mobile phase can also contain adjuvants, which may be added thereto in the formation of a one-time mixture, or may be added continuously or in time-dependent concentrations. Useful adjuvants include surfactants, sugars, salts, soluble organic compounds, buffers, and tris(hydroxymethyl)aminomethane. The mobile phase may alternatively be comprised of water containing ions, ionic liquids, conducting liquids, semiconducting liquids, or mixtures thereof.

Nucleic acid molecules suitable for use for forming hybrids may be of any type and from any source and include but are not limited to DNA, RNA and peptide nucleic acids. The nucleic acid molecules may be either single stranded or double stranded and may optionally be functionalized at any point with a variety of reactive groups, ligands or agents. The nucleic acid molecules may be generated by synthetic means or may be isolated from nature by protocols known from sources such as Sambrook, Fritsch and Maniatis, Molecular Cloning: A Laboratory Manual, Second Edition, Cold Spring Harbor Laboratory Press, Cold Spring Harbor, N.Y. (1989) (hereinafter “Sambrook”); Silhavy, Bennan and Enquist, Experiments with Gene Fusions, Cold Spring Harbor Laboratory Cold Press Spring Harbor, N.Y. (1984); and Ausubel et al, Current Protocols in Molecular Biology, published by Greene Publishing Assoc. and Wiley-Interscience (1987). Methods for the synthesis and use of PNAs are known from sources such as Antsypovitch, Peptide nucleic acids: Structure, Russian Chemical Reviews (2002), 71(1), 71-83.

Functionalization of the nucleic acid molecules used herein is typically not necessary for their association with CNTs to disperse the CNTs and form hybrids. Functionalization may, however, be of interest after the CNTs have been dispersed and it is desired to bind other moieties to the nucleic acid or immobilize a hybrid formed from a CNT-nucleic acid complex to a surface through various functionalized elements of the nucleic acid. The nucleic acids that are used for dispersion thus typically lack functional groups and are referred to herein as unfunctionalized.

Nucleic acid molecules as used herein may have any composition of bases, and may even consist of chain segments of varying length of the same base (polyadenosine or polythyamine, for example) without impairing the ability of the nucleic acid molecule to disperse bundled CNTs. The nucleic acid molecules may have less than about 2000 bases, or less than 1000 bases; or may have from about 5 bases to about 2000 bases, or about 5 bases to about 1000 bases. Generally the ability of nucleic acids to disperse CNTs appears to be independent of sequence or base composition. In certain embodiments, however, a lower amount of G-C and T-A base-pairing interactions in a sequence may provide a higher dispersion efficiency, or RNA and varieties thereof may be particularly effective in dispersion.

In addition to the combinations listed above, any of these sequences may have one or more deoxyribonucleotides replaced by ribonucleotides (i.e. RNA or RNA/DNA hybrid), or may have one or more sugar-phosphate linkages replaced by peptide bonds (i.e. PNA or PNA/RNA/DNA hybrid).

Single-stranded DNA (ssDNA) oligonucleotides suitable for use herein may be obtained from Integrated DNA Technologies, Inc. (Coralville Iowa), and yeast tRNA may be obtained from Sigma (St. Louis Mo.). RNA homopolymers poly(A), poly(C) and poly(U) suitable for use herein may be obtained from Amersham Biosciences (Piscataway N.J.). The letters “A”, “G”, “T”, “C” when referred to in the context of nucleic acids will mean the purine bases adenine (C₅H₅N₅) and guanine (C₅H₅N₅O), and the pyrimidine bases thymine (C₅H₆N₂O₂) and cytosine (C₄H₅N₃O), respectively.

Hybrids of CNTs and nucleic acid molecules may be obtained, for example, from a preparation as described below, which was assembled as follows: 10 mg of CNTs were suspended in 10 mL of 1×SSC buffer (0.15M NaCl and 0.015M sodium citrate), and then sonicated for 2 minutes with a TORBEO 130-Watt Ultrasonic Processor from Cole-Parmer Instrument Company (Vernon Hills Ill.). Nucleic acids were dissolved in H₂O to give a final concentration of 10 mg/mL. 50 μL of the CNT suspension and 5 μL of 10 mg/mL nucleic acid solution were added to 200 μL of H₂O to give a final volume of 255 μL. The mixture was sonicated for 3 minutes, followed by 90 minutes of centrifugation at 16,000 g with a Biofuge Fresco from Kendro Laoratory Products (Newtown Conn.). The supernatant was then removed for spectroscopic measurement. Absorption spectra from 400 nm to 900 nm were recorded using an Ultrospec 3300 UV-Vis spectrophotometer from Amersham Biosciences (Piscataway N.J.). The 730 nm peak was taken as a measure of the yield of the dispersion process.

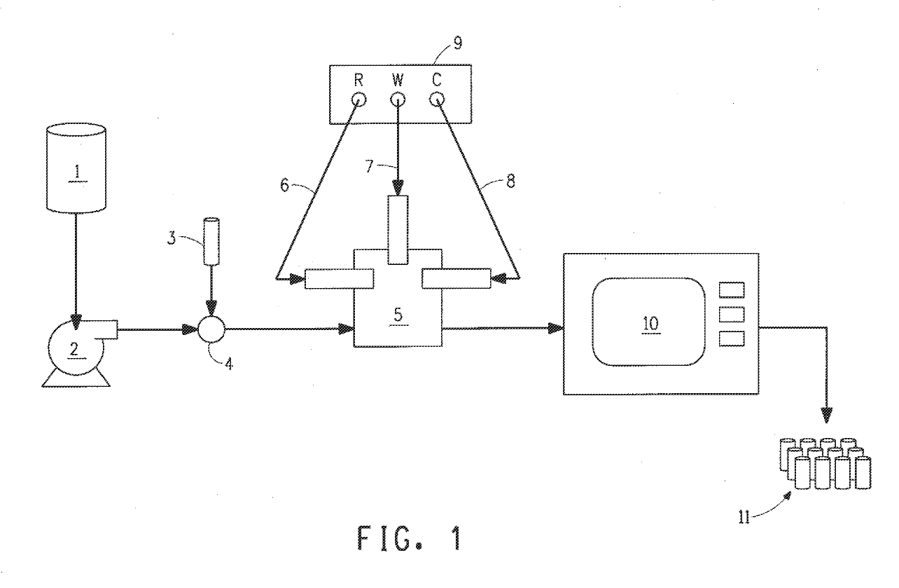

The methods hereof for separating charged species may be performed by an apparatus hereof, and one embodiment of an apparatus hereof, which is also an apparatus suitable for performing a method of separation hereof, is shown in FIG. 1. The apparatus shown in FIG. 1 includes a mobile phase reservoir (1), which is attached by tubing to a pump (2). The pump (2) is in turn attached by tubing to, and transports fluid to, an injector (4) such as an Agilent 1100 series HPLC. Also attached to the injector (4) by tubing is a sample reservoir (3) from which sample may be withdrawn and transported to the injector (4).

The injector (4) is connected by tubing to an electrochemical cell (5). The injector (4) together with the pump (2) serve to insert an aliquot of sample into a flowing stream of mobile phase fluid and deliver that mixture to the electrochemical cell (5). The electrochemical cell (5) has an interior volume into which projects a flat glass slide that serves as the working electrode (7). The working electrode can be plated with a conductive metal such as gold. The working electrode can also be a vibrating quartz crystal microbalance, which, in such embodiment, permits monitoring the weight of adsorbed and desorbed hybrids in real time for optimizing separations of hybrids.

Various different coatings are suitable for use on the working electrode, examples of which include metallic coatings such as gold, iron, platinum, copper or aluminum. The coatings may also be insulating materials such as polymer(s), alumina or other metal oxides, proteins, nucleic acids, and glass or other ceramics. The coatings may also be semiconductors such as mixtures formed from elements such as gallium, arsenic, germanium, indium and tin.

In addition to at least one working electrode (7), the electrochemical cell (5) further includes a reference electrode (6) and a counter electrode (8), both of which project into the interior volume of the cell and which are in contact with the flowing mobile phase. The electrochemical cell is macroscopically large, and it presents a non-tortuous flow path through its interior volume such that large hybrids may readily flow through it without becoming permanently entangled or entrapped in the same manner as might occur in a chromatography column filled with dense packing.

All three electrodes are all connected to an electrical source, such as a potentiostat (9) [such as may be obtained from Applied Princeton Research (Princeton, N.J.)], which supplies an electrical potential at its terminal “W” to the working electrode (7). As shown in FIG. 1, the potentiostat (9) is also connected, via terminals “R” and “C”, to the reference and counter electrodes (6) and (7), respectively. The electrochemical cell (5) is connected by tubing to an analytical monitor (10) that can detect and/or measure optical, chemical and/or electrical properties of the fluid flow. Fluid may be transported through the tubing from the cell (5) to the analytical monitor (10).

Examples of a suitable analytical monitor include a diode array detector, a refractive index detector, a thermal conductivity detector, a fluorescence detector, a mass spectrometer detector, a UV detector, a VIS detector, and a UV/VIS detector. Where the analytical monitor is a liquid chromatograph, the invention provides a unique combination of a liquid chromatograph and a potentiostat.

Mobile phase fluid containing a sample of hybrids is flowed through the apparatus by the action of the pump (2) and the injector (4). Mobile phase fluid is fed from reservoir (1) and is passed from the pump through tubing that is connected to the injector, and a sample of hybrids and fluid is inserted from sample container (3) by the injector (4) into the tubing and is passed on to the cell (5). As the hybrids are negatively charged, they adsorb on the positively charged anode, which is the working electrode (7). As the potential on the working electrode is changed, hybrids are desorbed from the working electrode, and they are thereafter detected in the flowing mobile phase using the analytical monitor. As the hybrids exit the analytical monitor, they are separated in different fractions by a fractionator as a collection device (11).

As noted above, all three electrodes are connected to a potentiostat (9), which makes it possible to control and vary the electrochemical potential on the working electrode. At the time of sample injection, the working electrode (7) potential may, for example, be set to a charge that is opposite the charge of the hybrid, which would typically result in a potential at the working electrode of about +0.9V. The hybrids, which are negatively charged at multiple sites (i.e. polyanionic), are consequently attracted to and adsorbed on the surface of the working electrode. Although the electrochemical cell (5) may not adsorb all hybrids flowing through it, those that pass through the cell and are not adsorbed are recovered as they exit the analytical monitor and flow into the fractionator (11).

Hybrids that are adsorbed on the surface of the working electrode (7) are differentially released therefrom as the potential on the working electrode is varied, which variation typically ranges from about +0.9V to about −0.9V. The electrical potential on the working electrode may be changed in any convenient time-varying manner. For example, the electrical potential may be changed in discrete steps or be continuously ramped. The highest conductivity hybrids develop the lowest magnitude of negative charge, are the least strongly attracted to and adsorbed on the working electrode, and are thus the first to desorb from the working electrode as the magnitude of the positive charge on the working electrode is reduced.

As seen from the foregoing, the electrical source, such as the potentiostat, is operable alternatively
to impose on the working electrode at least a first predetermined voltage referenced with respect to the reference electrode sufficient to cause some of the species in the dispersion to adsorb onto the working electrode and, thereafter,
to impose on the working electrode at least a second predetermined voltage also referenced with respect to the reference electrode sufficient to cause some of the adsorbed species to desorb from the working electrode and into the mobile phase.

The counter electrode also electrically contacts a mobile phase being pumped through the cell and is operable to inject an electric current into the mobile phase to maintain the voltage imposed on the working electrode substantially constant.

In an alternative embodiment, the working electrode may be given a negative charge, which will attract cations of various salt species present in the mobile phase. The negatively charged hybrids are then attracted to the cations bound to the negatively-charged working electrode, creating a double-layer effect on the working electrode.

Hybrids may also be formed by the binding of polycations to the carbon nanotubes. In such embodiment, similar separation strategies corresponding to those described above apply for binding positively-charged hybrids to a negatively-charged working electrode, or to a positively-charged working electrode with bound anions from the mobile phase.

The methods hereof further involve steps to collect hybrids with the mobile phase. The hybrids are contained in the mobile phase, which passes the working electrode at any given potential. The hybrids may be isolated from the mobile phase on a collection device (11) by evaporation of the fluid or filtration. For the purpose of the examples, the hybrids in the mobile phase are analyzed in a diode array detector (10) to determine the adsorption peak wavelength, which is in turn related to the conductivity of the hybrids.

In other embodiments of the methods hereof, where a nucleic acid molecule used in forming a hybrid has been functionalized with a first member of a binding pair, the hybrid may be immobilized on a solid support containing a second member of the binding pair by means of the mutual attraction of the members of the binding pair causing the nucleic acid to be deposited on the solid support.

Alternatively it will be possible to immobilize a hybrid as used herein by direct interaction between nucleic acid molecules. For example, nucleic acid molecules in the dispersion sample may be chosen or designed to incorporate a specific domain or sequence that will associate with a specific complementary sequence. Nucleic acids having the complementary sequence to the domain of the hybrid may be placed on the surface of a support, and the hybrid captured by the action of that association.

The above mentioned interactions that enable the immobilization of a hybrid as used herein may be equally employed to associate individual hybrids with other hybrids in a specific fashion. For example, a biotin containing hybrid may associate with a streptavidin containing hybrid, or a domain of one nucleic acid molecule may be designed to bind to a similar domain on another hybrid. In this fashion, the various hybrids may be rationally associated or immobilized to facilitate device fabrication.

The term “binding pair” refers to chemical or biopolymer based couples that bind specifically to each other. Common examples of binding pairs are immune-type binding pairs, such as antigen/antibody or hapten/anti-hapten systems. Suitable binding pairs glutathione-S-transferase/glutathione, 6× histidine Tag/Ni-NTA, biotin/avidin, streptavidin/biotin, S-protein/S-peptide, cutinase/phosphonate inhibitor, antigen/antibody, hapten/anti-hapten, folic acid/folate binding protein, and protein A or G/immunoglobulins. Another example of a binding pair is a negatively charged phosphate backbone of a nucleic acid molecule, with the second member being a positively charged surface. In the binding pairs, a “ligand” or “reactive ligand” will refer to one member of a binding pair which has been incorporated into the nucleic acid, and may include but is not limited to antibodies, lectins, receptors, binding proteins or chemical agents.

Methods for incorporating binding pair members onto the surface of solid supports are also known from sources such as Immobilized Enzymes, Inchiro Chibata, Halsted Press, New York (1978); and Cuatrecasas, J. Bio. Chem., 245: 3059 (1970).

A “solid support” is a material suitable for the immobilization of a CNT-nucleic acid hybrid. Typically the solid support provides an attachment of a member of a binding pair through which the complex is capture and immobilized. Solid supports suitable for such purposes are known in the art and include without limitation silicon wafers; synthetic polymer supports such as polystyrene, polypropylene, polyglycidylmethacrylate, substituted polystyrene (e.g. aminated or carboxylated polystyrene), polyacrylamide, polyamide or polyvinylchloride; glass; agarose; nitrocellulose; nickel grids or disks; silicon wafers; carbon supports; aminosilane-treated silica; polylysine coated glass; mica; and semiconductors such as Si, Ge and GaAs.

Immobilization of nucleic acids to a solid support is known in the art and may be accomplished for example using ultraviolet irradiation, baking, capillary transfer or vacuum transfer. Examples of nucleic acid immobilization on nitrocellulose and other suitable supports are given in Kalachikov et al, Bioorg. Khim., 18, 52 (1992); and Nierzwicki-Bauer et al, Biotechniques, 9, 472, (1990).

In an alternative embodiment of a method hereof, the flow rate of the mobile phase in the flow cell 5 may be varied. This can be done by varying the speed of the pump 2. The flow rates may range from about 1 to about 10,000 micro-liters per minute. The preferred range of flow rate is about 25 to about 300 micro-liters per minute.

In a further alternative embodiment of a method hereof, the composition of the mobile phase in the flow cell 5 may be varied. This can be done by adding salts or other adjuvants as described above to the mobile phase. The addition of the salts or other adjuvants may be step-wise, pulsed, or ramped.

In a further alternative embodiment of a method hereof, the temperature of the working electrode 7 in the flow cell 5 may be varied. This can be done by heating or cooling the working electrode 7 by, for example, a convection oven, a thermo-electric device, by radiation, or a heating coil. The temperature may range from about 5° C. to about 95° C. The preferred range of temperature is about 15° C. to about 40° C.

“Agitation means” as used herein to facilitate separation refers to a device that increases the dispersion of nanotubes and nucleic acids. A typical agitation means is sonication. The collection device (11) used herein may, for example, be a fractionator that deposits individual, selected portions of the flow of mobile phase passing out of the analytical monitor in separate locations by connecting the tubing through which the flow is passing to a robotically controlled arm, where the arm is moved over a series of open vials and stops over a selected vial to deposit the flow of mobile phase therein until instructed to move to another vial according to a signal related to the passage of time, a change in the voltage applied to the working electrode, or to a change in the output of the analytical monitor.

As the methods hereof do not rely on the use of an elution salt, the fractionated hybrids do not require additional purification. Furthermore, the nucleic acid molecules within each fractionated hybrid sample can be readily separated from the CNTs by adding an alkaline earth metal salt to the hybrid fraction and recycling the DNA for further separations. A separation of CNTs, as the charged species herein, is thus provided.

Once collected, the hybrids may be either immobilized on a solid substrate or rationally associated with other complexes in a process of nano-device fabrication. This invention can be used to supply CNTs with a homogenous set of electronic properties for applications that depend on components having predictable, homogenous electronic properties, which include the use of CNTs in devices such as sensors, electron field emitters or transistors.

### EXAMPLES

The single wall carbon nanotubes used in these examples were made by the HiPCO process, either purified or unpurified, and were purchased from CNI (Houston, Tex.). The materials were used as received without further modification. The various embodiments of the invention are not limited to those shown in the examples.

### DNA Dispersion of Nanotubes:

To produce a dispersion of hybrids 1 mg of nanotubes, produced via the HiPCO process was suspended in 1 ml aqueous DNA solution (1 mg/ml-ssDNA, 0.1M NaCl). The mixture was kept in an ice-water bath and sonicated (Sonics, VC130 PB) for 90 min at a power level of 3 W. After sonication, the samples were divided into 0.1 ml aliquots, and centrifuged (Eppendorf 5415C) for 90 min at 16,000 G to remove insoluble material leaving DNA-dispersed nanotube solutions at a mass concentration in the range of 0.2 to 0.4 mg/ml. The solution was then diluted by a factor of 50.

### Working Electrode Substrates:

Indium-Tin-Oxide (ITO) wafers were purchased from SPI Supplies. Gold coated substrates were created on microscope slides via e-beam deposition of a 5 nm titanium binder layer followed by 50 nm layer of gold at a deposition rate of 0.7 to 1.5 Angstroms/second.

### Flow Cell:

All experiments were done using an acrylic flow cell purchased from ICMFG modified to include a reference electrode. A platinum counter electrode is mounted at the flow cell top and an Ag/AgCl reference electrode is mounted along the outflow line. FIG. 1 shows the basic schematic. The reference electrode is threaded and thus holds a tight seal when an O-ring is added to the base of the threads. The counter electrode is held in by a mounted plate and sealed tightly with O-rings. The counter electrode protruded barely past the top entrance of the flow cell.

### Additional Equipment:

An HPLC (Series 1100, Agilent) accurately controlled the flow rate, pressure, and concentrations of all materials going through the flow cell. The diode array detector has a detection range of 190-950 nm range and was used to monitor the SWCNT absorption peaks as they moved through the system. A potentiostat (Princeton Applied Research model 283) was used to control the surface electrical potential on the metallic wafer working electrode relative to the is reference electrode.

The sample materials within the flow cell may either become adsorbed and desorbed, or simply flow free of capture by the active working electrode. All materials can be collected by a fractionator based on the DAD signal. This collected material can then be recycled into the injection sample to iteratively change the starting SWCNT population in a new injection.

A Diode Array Detector is used to determine the chirality or group of chiralities being ejected from the flow cell. Every SWNT chirality has a well-defined optical signature detected in the detector. Any deviation in the ratio of the observed detected peaks is must be due to a physical change in the ratio of constituent SWNTs present. Therefore as long as the signal/noise ratio is high, changing in the relative peak heights is a clear sign of enrichment.

### Methods

### Surface Preparation:

Gold coated wafers were cleaned stepwise by soaking in isopropyl alcohol, rinsing with 18.3 MegaOhm water, soaking for 10 minutes in Piranha solution (a 60:40 mixture of concentrated sulfuric acid and hydrogen peroxide), then rinsed again with 18.3 MegaOhm water. ITO wafers were only cleaned by rinsing in isopropyl alcohol and rinsing in 18.3 MegaOhm water to prevent etching and/or destruction of the surface by an acid.

### Standard Experimental Protocol:

In the following examples, 5 μL of ssDNA/SWCNT hybrid mixture solution was injected into the flow line of 18.3 MegaOhm water mobile phase as the surface potential at the working electrode is held at the trapping voltage. Then the working voltage was either stepped, ramped or switched to the releasing voltage profile as indicated in the specific examples below. After the experiment is over, a cleaning cycle is run to eject any remaining materials off the surface and prepare for the subsequent experiments. A cleaning cycle involves rapidly changing the potential between −900 mV and 900 mV over 30 seconds while increasing the flow rate to 100-300 μL/min to quickly whisk the materials away. The entire process (experiment and clean cycle) runs about 12 minutes.

### Data Analysis:

The spectra recorded by the diode array detector characterize the concentrations of the various hybrid chiralities passing through the detector at different times during the experiment. These data measure the relative amounts of a given hybrid chirality that either bypass the flow cell, i.e. were not initially adsorbed at the working electrode after injection, or become adsorbed and eventually desorbed at the working electrode after injection. A given hybrid chirality has its unique absorption at a wavelength, λ. Therefore the release ratio, R_{λ,t}_{₁}_{,t}_{₀}, is defined for each characteristic hybrid absorption wavelength as the ratio of the quantity which is both adsorbed and desorbed at the working electrode at time t₁ to the quantity which bypasses the working electrode at an earlier time t₀.

R_{λ,t}_{₁}_{,t}_{₀}=[Release_{λ,t}_{₁}]/[Bypass_{λ,t}_{₀}]

Since the hybrid extinction coefficient is invariant to these two populations of the same hybrid chirality, the ratio of the spectral absorbances is exactly equal to the ratio of the actual molar concentrations. The enrichment factor, E_{λ,t}_{₁}_{,t}_{₀}, is defined for each characteristic hybrid absorption wavelength as the release ratio for a given release time t₁ and bypass time t₀ divided by the smallest observed release ratio for the same times over the entire population of hybrids present in the sample.

$${E}_{λ , {t}_{1} , {t}_{0}} = {R}_{λ , {t}_{1} , {t}_{0}} / {min}_{all {λ}^{′}} ( {R}_{{λ}^{′} , {t}_{1} , {t}_{0}} )$$

Since the release ratio measures the exact ratio of molar concentrations released by the working electrode and bypassing the electrode, the enrichment factor indicates how well the adsorb-bypass process purifies any single hybrid chirality population relative to all other hybrid populations present in the sample. An enrichment ratio value of unity indicates no enrichment of a given hybrid chirality occurs since all hybrid populations were adsorbed and desorbed at the same relative concentrations at the times of measurement. However, when a process separates one hybrid chirality in the mixture by the adsorb/desorb mechanism, this hybrid enrichment factor will exceed unity. The enrichment factor grows larger from unity as the separation and purification of this given hybrid from the mixture increases. The adsorb/desorb process can be repeated any number of times desired for any enriched fraction of hybrids with negligible loss of material using the injector, flow cell and fractionator.

The purity, P_{λ,t}_{₁}_{,t}_{_{0.N}}, expressed as a mole fraction obtained by repeating the enrichment N-times is given simply by

$${P}_{λ , {t}_{1} , {t}_{0 , N}} = \frac{{{C}_{λ}^{0} ( \frac{{R}_{λ , {t}_{1} , {t}_{0}}}{1 + {R}_{λ , {t}_{1} , {t}_{0}}} )}^{N}}{{\sum }_{{λ}^{′}} {{C}_{{λ}^{′}}^{0} ( \frac{{R}_{{λ}^{′} , {t}_{1} , {t}_{0}}}{1 + {R}_{{λ}^{′} , {t}_{1} , {t}_{0}}} )}^{N}}$$

where C_{λ}⁰ is the initial concentration of hybrid absorbing at characteristic wavelength λ. Therefore a desired level of purity can be obtained from a separation process having an enrichment factor greater than unity by fractionating aliquots of eluant at different times and repeating the enrichment. Furthermore fractions from different separation conditions may be repeated to isolate any desired hybrid chirality once an enrichment condition has been discovered for the desired hybrid chirality.

### Examples 1-6

In Examples 1-6 all experimental conditions were the same as described above, except where noted in the following table.

| Example | Elution Conditions |
|---|---|
| 1 | The working electrode surface was gold. The working electrode |
| | potential was set to −900 mV for adsorption and step-switched to |
| | +900 mV for desorption while the mobile phase flow rate was 300 |
| | μL/min. |
| 2 | The working electrode surface was gold. The working electrode |
| | potential was set to −900 mV for adsorption and ramped to +600 mV at |
| | a rate of 6 mV/sec for desorption while the mobile phase flow rate was |
| | 25 μL/min. |
| 3 | The working electrode surface was gold. The working electrode |
| | potential was held in four periods at −900/−900/900/900 mV for |
| | 2/2/2/2 minutes at mobile flow rates of 50/300/50/50 μL/min. |
| 4 | The working electrode surface was gold. The working electrode |
| | potential was held in three periods at −900/900/0 mV for 4/3/3 |
| | minutes at a constant mobile phase flow rate of 50 μL/min |
| 5 | The working electrode surface was ITO. The working electrode |
| | potential was set to −900 mV for 6 minutes and then step switched to |
| | 900 mV for 3 minutes during a constant mobile phase flow rate of 25 |
| | μL/min |
| 6 | The working electrode surface was ITO. The working electrode |
| | potential was set to 900 mV for 6 minutes and then step switched to |
| | −900 mV for 3 minutes during a constant mobile phase flow rate of 25 |
| | μL/min |

The following enrichment factors were noted for each SWNT chirality (m,n) absorbing at wavelength, A, in each of Examples 1˜6:

| | | Enrichment Factors, E_{λ} | | | | | |
|---|---|---|---|---|---|---|---|
| (m, n) | λ, nm | #1 | #2 | #3 | #4 | #5 | #6 |
| 8, 2 | 406 | 1.402 | 1.000 | 1.000 | 1.000 | 1.150 | 1.115 |
| 7, 4 | 460 | 1.000 | 1.085 | 1.166 | 1.067 | 1.081 | 1.062 |
| 9, 3 | 510 | 1.854 | 1.149 | 1.248 | 1.104 | 1.031 | 1.008 |
| 6, 4 | 570 | 2.195 | 1.170 | 1.167 | 1.030 | 1.044 | 1.008 |
| 6, 5 | 574 | 2.220 | 1.170 | 1.153 | 1.037 | 1.050 | 1.015 |
| 8, 4 | 589 | 2.244 | 1.170 | 1.247 | 1.022 | 1.050 | 1.023 |
| 7, 6 | 648 | 2.427 | 1.128 | 1.220 | 1.096 | 1.025 | 1.008 |
| 7, 5 | 654 | 2.500 | 1.106 | 1.074 | 1.015 | 1.125 | 1.085 |
| 8, 3 | 670 | 2.451 | 1.170 | 1.288 | 1.030 | 1.031 | 1.008 |
| 9, 1 | 680 | 2.427 | 1.170 | 1.313 | 1.022 | 1.013 | 1.000 |
| 8, 7 | 712 | 2.427 | 1.149 | 1.352 | 1.044 | 1.000 | 1.000 |

In other embodiments hereof, the separation methods of this invention could be performed in a manner as would be obtained from the following Illustrative Embodiments A and B:

### Illustrative Embodiment A

Metallic-type SWNTs (8,2), (7,4) and (9,3) are separated from semiconducting-type SWNTs (6,4), (6,5), (8,4), (7,6), (7,5), (8,3), (9,1), (8,7) from their initial mixture. The initial mixture is subject to the adsorb/desorb conditions as described in Example 1. The by-passed fraction is collected first in one vial and found to be enriched in the metallic SWNTs. The released fraction is collected next in a second vial and found to be enriched in the semiconducting SWNTs. The contents of the initial first vial fraction is again subject to adsorb/desorb conditions as described in Example 1; the bypassed fraction is collected in one vial and found to be enriched in the metallic SWNTs; and the released fraction is collected in a second vial and found to be enriched in the semiconducting SWNTs. The contents of the initial second vial fraction is again subject to adsorb/desorb conditions as described in Example 1; and the bypassed fraction is collected in one vial and found to be enriched in the metallic SWNTs; and the released fraction is collected in a second vial and found to be enriched in the semiconducting SWNTs.

Now all fractions enriched in metallic SWNTs are combined into a first generation metallic-enriched vial, and all fractions enriched in semiconductiong SWNTs are combined into a first generation semiconducting-enriched vial. The first generation metallic-enriched vial population is now again subject to the adsorb/desorb conditions as described in Example 1, and the metallic enriched fraction and the semiconducting enriched fraction are separated into second generation vials. The first generation semiconducting-enriched vial population is now again subject to the adsorb/desorb conditions as described in Example 1, and the metallic enriched fraction and the semiconducting enriched fraction are separated into second generation vials.

This process is repeated with the second generation metallic-enriched fraction and the second generation semiconducting-enriched fraction to form a third generation metallic enriched fraction and a third generation semiconducting enriched fraction. The process is repeated until the sixth generation where the metallic fraction is sensibly free of semiconducting SWNTs, and the semiconducting fraction is sensibly free of metallic SWNTs.

### Illustrative Embodiment B

The semiconducting fraction as would be obtained from Example 7 is now subjected to the separation conditions described in Example 6, and the bypass fraction is found to be enriched in (6,4), (6,5), (7,6), (8,3), (9,1) and (8,7) SWNTs while the released fraction is found to be enriched in (8,4) and (7,5) SWNTs. Both fractions are repeatedly enriched using the conditions as described in Example 6 for ten generations of enrichments until the tenth generation desorbed fraction contains sensibly only (8,4) and (7,5) SWNTs. This fraction is now separated using the conditions described in Example 5 wherein the bypassed fraction is enriched in (8,4) SWNTs, and the desorbed fraction is enriched in (7,5) SWNTs. Both fractions are repeatedly enriched using the conditions in Example 6 for fifteen generations of enrichments until the fifteenth generation desorbed fraction is sensibly pure in (7,5) SWNTs, and the fifteenth generation bypass fraction is sensibly pure in (8,4) SWNTs.

In yet other embodiments hereof, one or more of the methods of separation of this invention may be performed by an apparatus for separating a mixture of species dispersed in a mobile phase, at least some of the species having different electrical properties, wherein the apparatus includes (a) an electrochemical cell having an interior volume therein; (b) a delivery device for introducing a mobile phase having a mixture of species dispersed therein into the interior volume of the electrochemical cell; (c) the electrochemical cell having at least a first working electrode and a reference electrode both projecting into the interior volume, both the working electrode and the reference electrode being positioned in the cell to contact a mobile phase; (d) an electrical source connected to the working electrode, the source being operable alternatively
(i) to impose on the working electrode at least a first predetermined voltage referenced with respect to the reference electrode sufficient to cause some of the species in the dispersion to adsorb onto the working electrode and, thereafter,
(ii) to impose on the working electrode at least a second predetermined voltage also referenced with respect to the reference electrode sufficient to cause some of the adsorbed species to desorb from the working electrode and into the mobile phase.

In such an apparatus, the electrochemical cell may have an inlet port and an outlet port, and the delivery device may include a pump for pumping a mobile phase having a mixture of species dispersed therein at a predetermined flow rate through the interior volume of the electrochemical cell; and the interior volume of the cell can be a non-tortuous path through the interior volume of the electrochemical cell to the flow of a mobile phase and species dispersed therein.

Also in such an apparatus, the electrochemical cell may also include a collector for collecting the species desorbed from the working electrode. The electrochemical cell may also include a counter electrode projecting into the interior volume and being positioned to electrically contact a mobile phase being pumped through the cell, and the counter electrode can operate to inject an electric current into the mobile phase to maintain the voltage imposed on the working electrode substantially constant. The working electrode, the reference electrode and the counter electrode may be implemented and controlled using a potentiostat. The electrical source may be operated to impose the first and second voltages on the working electrode in accordance with a predetermined time-varying pattern, and the pump may be operated to pump a mobile phase through the cell at any of a plurality of flow rates.

Also in such an apparatus, the working electrode may have a metallic surface, or the working electrode may have a surface formed of an electrically insulating material. The apparatus may further include a heater for heating the working electrode, or a cooler for cooling the working electrode. The apparatus may also include a monitor disposed between the electrochemical cell and the collector for monitoring a predetermined characteristic of the species desorbed from the working electrode, such as a predetermined optical characteristic of the desorbed species, a predetermined chemical characteristic of the desorbed species, or a predetermined electrical characteristic of the desorbed species.

Such an apparatus may also include a second working electrode projecting into the interior volume and being positioned to contact a mobile phase being pumped therethrough; where the electrical source is connected to the second working electrode, and the electrical source is operable alternatively
to impose on the second working electrode a substantially constant voltage referenced with respect to the reference electrode sufficient to cause some of the species in the dispersion to adsorb onto the second working electrode and, thereafter,
to impose on the second working electrode a constant voltage also referenced with respect to the reference electrode sufficient to cause some of the adsorbed species to desorb from the second working electrode and into the mobile phase.

Particular methods of this invention may be performed by an apparatus for separating a mixture of nucleic-acid-hybridized carbon nanotubes dispersed in a mobile phase, at least some of the nanotubes having different electrical properties, where the apparatus includes (a) an electrochemical cell having an interior volume therein; (b) a pump for pumping a mobile phase having a mixture of nanotubes dispersed therein at a predetermined flow rate through the interior volume of the electrochemical cell; (c) in the electrochemical cell a first working electrode, a reference electrode, and a counter electrode all projecting into the interior volume, all of the electrodes being positioned in the cell to contact a mobile phase being pumped therethrough; (d) a potentiostat connected to the working, reference and counter electrodes, the potentiostat being operable (i) alternatively
(A) to impose on the working electrode at least a first predetermined voltage referenced with respect to the reference electrode sufficient to cause some of the nanotubes in the dispersion to adsorb onto the working electrode and, thereafter,
(B) to impose on the working electrode at least a second voltage also referenced with respect to the reference electrode sufficient to cause some of the adsorbed nanotubes to desorb from the working electrode and into the mobile phase; and/or
   (ii) to inject into the mobile phase through the counter electrode an electric current sufficient to maintain the voltages imposed on the working electrode at the predetermined values; (e) a collector for collecting nanotubes desorbed from the working electrode; and (f) a monitor disposed between the electrochemical cell and the collector for monitoring a predetermined characteristic of the nanotubes desorbed from the working electrode.

The term “invention” as used herein is a non-limiting term, and is not intended to refer to any single embodiment of the various inventions hereof to the exclusion of others, but encompasses all possible embodiments as described in the specification and the claims.

Where a range of numerical values is recited or established herein, the range includes the endpoints thereof and all the individual integers and fractions within the range, and also includes each of the narrower ranges therein formed by all the various possible combinations of those endpoints and internal integers and fractions to form subgroups of the larger group of values within the stated range to the same extent as if each of those narrower ranges was explicitly recited. Where a range of numerical values is stated herein as being greater than a stated value, the range is nevertheless finite and is bounded on its upper end by a value that is operable within the context of the invention as described herein. Where a range of numerical values is stated herein as being less than a stated value, the range is nevertheless bounded on its lower end by a non-zero value.

In this specification, unless explicitly stated otherwise or indicated to the contrary by the context of usage, where an embodiment of the subject matter hereof is stated or described as comprising, including, containing, having, being composed of or being constituted by or of certain features or elements, one or more features or elements in addition to those explicitly stated or described may be present in the embodiment. An alternative embodiment of the subject matter hereof, however, may be stated or described as consisting essentially of certain features or elements, in which embodiment features or elements that would materially alter the principle of operation or the distinguishing characteristics of the embodiment are not present therein. A further alternative embodiment of the subject matter hereof may be stated or described as consisting of certain features or elements, in which embodiment, or in insubstantial variations thereof, only the features or elements specifically stated or described are present.

Methods and apparatus for separating species in a mixture, such as a mixture of CNTs, are further described in U.S. Provisional Application 61/262,632, published as U.S. patent Publication Ser. No. ______, which is by this reference incorporated in its entirety as a part hereof for all purposes.

What is claimed is:

1. A method of partitioning a population of charged species comprising (a) providing a dispersion of the charged species in a liquid, (b) contacting the liquid dispersion with a first electrode to which is applied a voltage selected to attract and adsorb to the electrode a portion of the species contained in the liquid dispersion, (c) desorbing from the electrode some or all of the adsorbed species to provide a first fraction of species, and (d) extracting the first fraction of species from the population.

2. A method according to claim 1 wherein one or more charged species comprise a hybrid formed from a carbon nanotube and a nucleic acid molecule.

3. A method according to claim 1 wherein bundled carbon nanotubes are contacted with nucleic acid molecules to form the dispersion of charged species.

4. A method according to claim 1 wherein the voltage of the first electrode is adjusted to desorb adsorbed species from the first electrode.

5. A method according to claim 1 wherein the voltage of the first electrode is adjusted in a time-varying pattern.

6. A method according to claim 1 wherein the voltage of the first electrode is adjusted in discrete steps or in a continuous ramp.

7. A method according to claim 1 wherein the composition of the liquid dispersion is adjusted to desorb adsorbed species from the first electrode.

8. A method according to claim 1 wherein a stream of the liquid dispersion is flowed past the first electrode to contact the liquid dispersion with the first electrode, and the velocity of the stream is adjusted to desorb adsorbed species from the first electrode.

9. A method according to claim 1 wherein the species are negatively charged.

10. A method according to claim 1 wherein the species are positively charged.

11. A method according to claim 1 wherein the first electrode is negatively charged.

12. A method according to claim 1 wherein the first electrode is positively charged.

13. A method according to claim 1 further comprising determining the weight of the portion of species that are adsorbed on the first electrode.

14. A method according to claim 1 further comprising contacting the liquid dispersion with a second electrode to which is applied a voltage selected to attract and adsorb to the second electrode the same or a different portion of the species contained in the liquid dispersion than is attracted to the first electrode.

15. A method according to claim 14 wherein the voltage applied to the second electrode attracts and adsorbs on the electrode a different portion of the species contained in the liquid dispersion than is attracted to the first electrode.

16. A method according to claim 14 wherein the voltage applied to the second electrode attracts and adsorbs on the second electrode the same portion of the species contained in the liquid dispersion that is attracted to the first electrode; wherein the voltage of the first electrode is adjusted by a first amount to provide the first fraction of species; and wherein the voltage of the second electrode is adjusted by a second amount to provide a second fraction of species.

17. A method according to claim 1 further comprising analyzing the first fraction of species to determine a property thereof.

18. A method according to claim 17 wherein the property determined is an optical, chemical or electrical property of the species.

19. A method according to claim 1 wherein the desorbed species are passed through a diode array detector to determine a property thereof.

20. A method according to claim 1 further comprising directing the first fraction of species to a first preselected container in a fractionator to extract the first fraction of species from the population.

21. A method according to claim 1 further comprising evaporating or filtering the liquid dispersion containing the first fraction of species to recover the species from the liquid.

22. A method according to claim 21 further comprising immobilizing the first fraction of species on a solid support.

23. A method according to claim 1 further comprising
collecting the first fraction of species;
extracting from the population and collecting some or all of the species that are not attracted to the first electrode to provide a third fraction of species;
analyzing the first fraction of species to determine a property thereof that identifies them as enriched in Type I species;
analyzing the third fraction of species to determine a property thereof that identifies them as enriched in Type III species;
contacting the first fraction again with the first electrode to provide an adsorbed/desorbed fraction that is further enriched in Type I species; and
repeatedly contacting the desorbed fraction with the first electrode to provide fractions that are increasingly enriched in Type I species.

24. A method according to claim 1 further comprising
collecting the first fraction of species;
extracting from the population and collecting some or all of the species that are not attracted to the first electrode to provide a third fraction of species;
analyzing the first fraction of species to determine a property thereof that identifies them as enriched in Type I species;
analyzing the third fraction of species to determine a property thereof that identifies them as enriched in Type III species;
contacting the third fraction again with the first electrode to provide an non-attracted fraction that is further enriched in Type III species; and
repeatedly contacting the non-attracted fraction with the first electrode to provide fractions that are increasingly enriched in Type III species.
